(19) United States
(12) Patent Application Publication
(10) Pub. No.: US 2022/0409135 A1
(43) Pub. Date: Dec. 29, 2022

(54) ELECTRODE-EQUIPPED BAND AND WEARABLE APPARATUS

(71) Applicant: OMRON HEALTHCARE Co., Ltd., Kyoto (JP)

(72) Inventors: Reiji FUJITA, Kyoto (JP); Yasuhiro KAWABATA, Kyoto (JP); Kenji FUJII, Kyoto (JP); Naomi MATSUMURA, Kyoto (JP); Akito ITO, Kyoto (JP); Yuki SAKAGUCHI, Kyoto (JP)

(21) Appl. No.: 17/929,523

(22) Filed: Sep. 2, 2022

Related U.S. Application Data
(63) Continuation of application No. PCT/JP2021/004150, filed on Feb. 4, 2021.

(30) Foreign Application Priority Data
Mar. 10, 2020 (JP) 2020-040669

(51) Int. Cl. A61B 5/00; A61B 5/256

(52) U.S. Cl. CPC A61B 5/6831; A61B 5/256; A61B 5/02438

(57) ABSTRACT

An electrode-equipped band includes a first band section and a second band section extending along a longitudinal direction. The second band section includes a first stretchable section, an electrode, a second stretchable section, a ring member, a fold-back section, and a joining section. The first stretchable section has first stretchability along the longitudinal direction. The electrode is provided on a first surface of the first stretchable section. The second stretchable section overlaps with the first stretchable section on a side of a second surface of the first stretchable section, and has second stretchability that allows greater stretch along the longitudinal direction than the first stretchable section. The ring member is provided at a position corresponding to a band end along the longitudinal direction of the second band section, and may be connected to the first band section.

### CROSS-REFERENCE TO RELATED APPLICATIONS

This application is the U.S. national stage application filed pursuant to 35 U.S.C. 365(c) and 120 as a continuation of International Patent Application No. PCT/JP2021/004150, filed Feb. 4, 2021, which application claims priority to Japanese Patent Application No. 2020-040669, filed Mar. 10, 2020, which applications are incorporated herein by reference in their entireties.

### TECHNICAL FIELD

One aspect of the present invention relates to an electrode-equipped band and a wearable apparatus.

### BACKGROUND ART

For example, a heart rate measuring device disclosed in Patent Document 1 detects an electrocardiographic signal in a state where the heart rate measuring device is wrapped around a chest by a band. When the heart rate measuring device is formed into an annular shape, a stretchable strap and a non-stretchable belt, on which electrodes are disposed, are connected to each other in a circumferential direction of the band using a metal fitting. Since a large potential difference can be achieved by disposing electrodes on both sides of the heart in electrocardiographic signal detection in the chest, it is sufficient that the electrode interval in the non-stretchable belt is relatively short. Therefore, in the heart rate measuring device, a stretchable section can be provided to be relatively long, and the length of the strap can be easily adjusted.

### CITATION LIST; PATENT LITERATURE

Patent Document 1: JP 5441977 B

### SUMMARY OF INVENTION

### Technical Problem

A wearable apparatus such as a heart rate measuring device may be attached to a limb (upper limb or lower limb) using a band. At this time, when the number of electrodes disposed on the band is increased or the area of each electrode is increased in order to detect a smaller potential difference, it may be difficult to incorporate a portion having stretchability in the circumferential direction of the band. In this case, after attaching the wearable apparatus to a user by the band, the length of the band does not follow an increase in the diameter (circumferential length) of the portion to which the wearable apparatus is attached, which may cause discomfort to the user.

An object of the present invention is to provide an electrode-equipped band and a wearable apparatus capable of suppressing the occurrence of discomfort to a user in a state of being attached to an appropriate position.

### Solution to Problem

The present invention may adopt the following configurations in order to achieve the above-mentioned object.

An electrode-equipped band according to an aspect includes a first band section having a band shape and extending along a longitudinal direction, and a second band section provided in a first direction along the longitudinal direction with respect to the first band section and extending along the longitudinal direction. The second band section includes a first stretchable section having an electrical insulation property, an electrode being conductive, a second stretchable section, a ring member having a ring shape, a fold-back section, and a joining section. The first stretchable section has a band shape, has a first surface and a second surface opposite to the first surface, and has first stretchability along the longitudinal direction. The electrode is provided on the first surface of the first stretchable section. The second stretchable section overlaps with the first stretchable section on a side of a second surface of the first stretchable section, has a band shape, and has second stretchability that allows greater stretch along the longitudinal direction per unit length than the first stretchability of the first stretchable section. The ring member is provided at a position corresponding to a band end along the longitudinal direction of the second band section, and may be connected to the first band section. The fold-back section is provided between the first stretchable section and the second stretchable section, and is folded back at the ring member so that the first stretchable section and the second stretchable section overlap with each other. The joining section joins the second surface of the first stretchable section and the second stretchable section.

The longitudinal direction is a direction along an extending direction (circumferential direction) of the band. In the electrode-equipped band, for example, in one second band section, the second stretchable section having stretchability higher than that of the first stretchable section is overlapped on the second surface of the first stretchable section provided with an electrode on the first surface. The first stretchable section and the second stretchable section are continuous at the fold-back section between the first stretchable section and the second stretchable section. In a state where such a band is attached to a living body, the second stretchable section of the second band section stretches in the longitudinal direction (circumferential direction) in accordance with the movement of the living body at the position where the band is attached. The relative position between the fold-back section and the ring member is changed by stretch of the second stretchable section, and the length of the band is adjusted. Thus, the stretchability of the one second band section is adjusted in accordance with the movement of the user, and thus it is possible to suppress the occurrence of discomfort to the user in a state where the electrode-equipped band is attached to an appropriate position. The band is preferably attached to a limb (upper limb or lower limb), but may be attached to the chest, abdomen, waist, or the like. Since the electrode is provided on the first surface of the first stretchable section that allows lower stretch than the second stretchable section, signal detection using the electrode, treatment using the electrode, or the like can be performed on the user to whom the band is attached while the positional relationship between the living body and the electrode is maintained in a fixed state. The signal detection using an electrode refers to obtaining a signal from a living body, such as a heart rate or an electrical signal that becomes an electrocardiogram when transmitted through an atrium and a ventricle and written out as a waveform. This is a “test” using an electrode. “Treatment” using an electrode means that a weak current of, for example, 1200 Hz or less is applied to a skin surface to stimulate the skin to eliminate the stiffness of the muscles of the affected portion.

In one aspect, it is preferable that the first stretchable section includes a base section having third stretchability that allows greater stretch along the longitudinal direction than the first stretchability, the base section forming the first surface, and a stretchability adjustment member fixed to the base section and having fourth stretchability that allows lower stretch along the longitudinal direction than the third stretchability, the stretchability adjustment member being configured to adjust stretchability of the first stretchable section along the longitudinal direction so that the first stretchable section has the first stretchability, the stretchability adjustment member forming the second surface.

According to such a configuration, the stretchability in the longitudinal direction of the first stretchable section of the second band section can be adjusted to a state having first stretchability by the base section and the stretchability adjustment member.

In one aspect, it is preferable that the first stretchable section has low stretchability or non-stretchability along the longitudinal direction.

While the first stretchable section hardly stretches and the electrode is disposed in the first stretchable section, the positional relationship between the living body and the electrode can be maintained in the more reliably fixed state.

In one aspect, it is preferable that the fold-back section has first stretchability in the longitudinal direction.

The fold-back section can be integrally formed as the same configuration as the first stretchable section.

In one aspect, it is preferable that the fold-back section has second stretchability in the longitudinal direction.

The fold-back section can be integrally formed as the same configuration as the second stretchable section.

In one aspect, it is preferable that the first band section is folded back at the ring member toward a position outside the first band section to adjust a length of the first band section, and has a detachable connecting section on the outside of the first band section.

The first band section allows initial adjustment of the circumferential length of the band when the band for the wearable apparatus mounted on the living body is attached.

In one aspect, it is preferable that the first stretchable section of the second band section includes a wire electrically connecting the electrode and a main body section provided in the electrode-equipped band.

Since there is a wire in the first stretchable section having less stretchability than the second stretchable section, it is possible to suppress a load applied in the longitudinal direction of the wire.

In one aspect, the wearable apparatus includes the electrode-equipped band, and a main body section provided between the first band section and the second band section of the electrode-equipped band and incorporating a control substrate electrically connected to the electrode.

Therefore, it is possible to provide a wearable apparatus capable of suppressing the occurrence of discomfort to a user in a state of being attached to an appropriate position.

For example, the main body section having a sensor, a transducer, or the like can be attached to an appropriate position of the electrode-equipped band. Therefore, the main body section having a desired function can be attached to the electrode-equipped band. For example, the heart rate can be measured by attaching a main body section having a function of measuring the heart rate to one band. Instead of the main body section having the function of measuring the heart rate, a main body section for detecting an electrocardiographic signal can be attached to one band to detect the electrocardiographic signal. It is possible to perform low-frequency treatment by attaching a main body section having a function of performing low-frequency treatment to one band. In this manner, various main body sections can be attached to one band and used.

### Advantageous Effects of Invention

According to the present invention, it is possible to provide an electrode-equipped band and a wearable apparatus capable of suppressing the occurrence of discomfort to a user in a state of being attached to an appropriate position.

### BRIEF DESCRIPTION OF DRAWINGS

The following description is the invention will be described and explained with additional specificity and detail through the use of the accompanying drawings, in which:

FIG. 1 is a schematic perspective view illustrating a wearable apparatus according to a first embodiment;

FIG. 2 is a schematic perspective view illustrating the wearable apparatus viewed from a direction different from that of FIG. 1;

FIG. 3 is a schematic block diagram illustrating the wearable apparatus according to the first embodiment;

FIG. 4 is a schematic top view illustrating the wearable apparatus according to the first embodiment;

FIG. 5 is a schematic view of the wearable apparatus in FIG. 4 viewed from a direction indicated by an arrow V;

FIG. 6 is a schematic view of the wearable apparatus in FIG. 5 viewed from a direction indicated by an arrow VI;

FIG. 7 is an enlarged view of a position indicated by reference sign VII in FIG. 5;

FIG. 8 is a schematic view illustrating the wearable apparatus according to the first embodiment;

FIG. 9 is a schematic view illustrating an attachment of the wearable apparatus according to the first embodiment whose main body section is attached to a user in a state of facing the front of the user when the user is located in a normal anatomical position;

FIG. 10 is a schematic top view illustrating a wearable apparatus according to a second embodiment;

FIG. 11 is a schematic view of the wearable apparatus in FIG. 10 viewed from a direction indicated by an arrow XI;

FIG. 12 is a schematic view of the wearable apparatus in FIG. 11 viewed from a direction indicated by an arrow XII;

FIG. 13 is a schematic view illustrating the wearable apparatus according to the second embodiment;

FIG. 14 is a schematic view illustrating a wearable apparatus according to a third embodiment; and,

FIG. 15 is a schematic view illustrating an electrode-equipped band for a wearable apparatus according to a fourth embodiment.

### DESCRIPTION OF EMBODIMENTS

With reference to the drawings, embodiments of an electrode-equipped band 12 including electrodes 68a, 68b and a wearable apparatus 10 will be described.

### Application Example

The electrode-equipped band 12 including the electrodes 68a, 68b is attached to an appropriate position such as an appropriate limb (upper limb or lower limb), chest, abdomen, or waist of a user's body B. The band 12 may also be attached to a user's head. The band 12 is used with a main body section 14. The band 12 includes a first band section 62 whose length is largely adjusted (initial adjustment) in accordance with the attachment position of the user, and a second band section 64 in which a first stretchable section 112 and a second stretchable section 114 having different stretchability are disposed in an overlapping manner. A fold-back section 106 and a ring member 110 between the first stretchable section 112 and the second stretchable section 114 move in a circumferential direction (longitudinal direction) of the band 12 in accordance with the movement of the user at a position where the band 12 is attached to the user. Thus, the band 12 allows the movement of the user in the second band section 64, and prevents the movement from being hindered. The band 12 and the wearable apparatus 10 having the band 12 allow the user to move at the position where the band 12 is attached, thereby preventing the user from feeling uncomfortable.

The wearable apparatus 10 is configured to detect a signal from a living body such as a heart rate, an electrocardiographic signal, or a myoelectricity. Also, the wearable apparatus 10 may be configured to perform treatment by applying an electric signal of a low frequency, or the like to the living body.

### First Embodiment

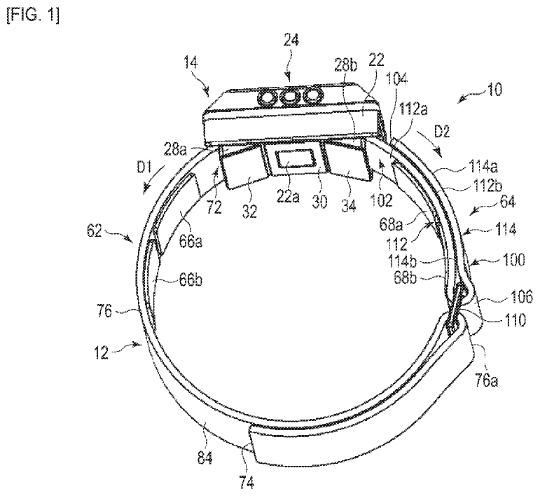

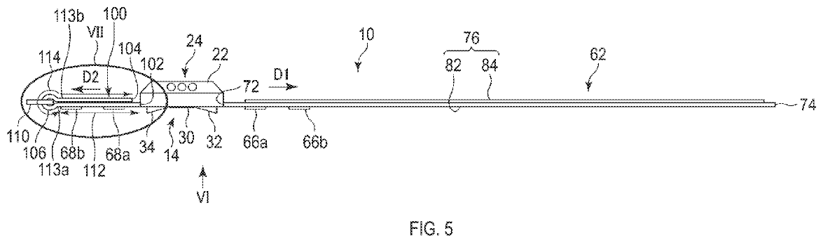

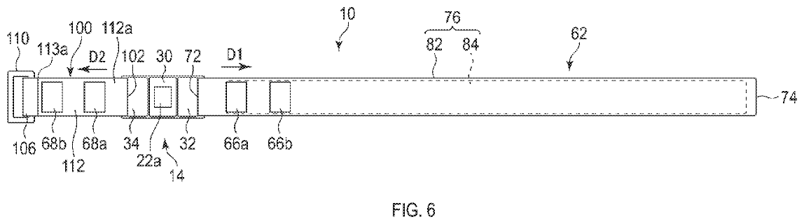

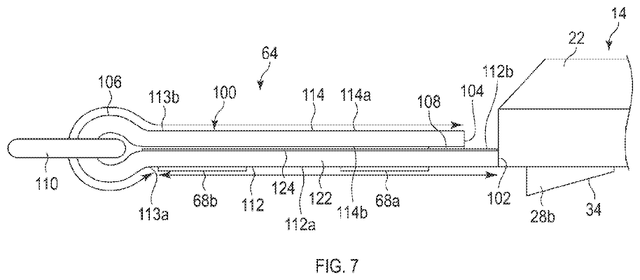

With reference to FIGS. 1 to 9, the wearable apparatus 10 and the electrode-equipped band 12 used together with the wearable apparatus 10 will be described. In the present embodiment, a case where the wearable apparatus 10 measures the heart rate and the electrocardiographic signal is described as an example.

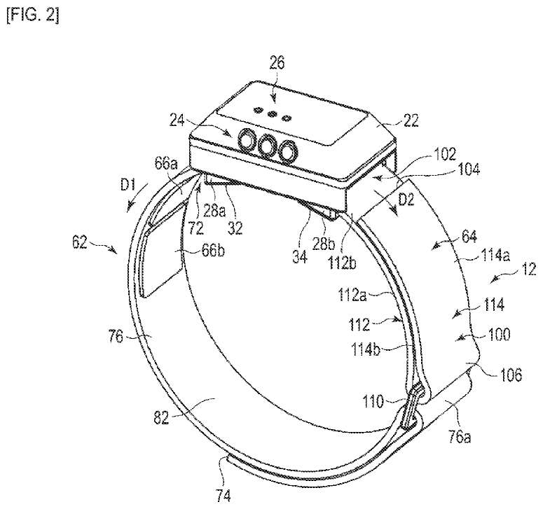

As illustrated in FIGS. 1 and 2, the wearable apparatus 10 has the electrode-equipped band (belt) 12 for the wearable apparatus 10 and the main body section 14 provided on the band 12.

In the wearable apparatus 10, the band 12 is fixed to the main body section 14. The wearable apparatus 10 including the band 12 and the main body section 14 is attached to, for example, a limb (upper limb or lower limb) of the user (living body) by the band 12. The band 12 is formed in a length that allows the band 12 to be attached to the limb of the user. The wearable apparatus 10 is formed in a size and weight that allows easy operation by the user while being attached to the user's limb by the band 12. When the wearable apparatus 10 is attached to the chest, waist, or head, the band 12 is formed in a length that allows the band 12 to be attached to the user's chest, waist, or head. Here, the wearable apparatus 10 is described as being attached to the upper left arm of the user.

The main body section 14 includes a frame (outer cover) 22, an operation unit 24 provided on the frame 22, a display unit 26 provided on the frame 22, a pair of support sections 28a, 28b provided on the frame 22, a first main body electrode (biological signal detecting unit) 30 provided on the frame 22, a second main body electrode (biological signal detecting unit) 32 provided on the support section 28a, and a third main body electrode (biological signal detecting unit) 34 provided on the support section 28b.

The frame 22 is formed of a material having an electrical insulation property. In a state where the wearable apparatus 10 is attached to the upper left arm of the user, the support sections 28a, 28b are located on the living body side (rear surface side) with respect to the frame 22, the operation unit 24 is located at a position where it can be operated by a finger, or the like of the user, and the display unit 26 is located at a position where it can be visually recognized by the user, that is, on the side (front surface side) opposite to the living body.

The support sections 28a, 28b are formed in a substantially wedge shape, for example. The support sections 28a, 28b protrude from the rear surface of the frame 22, and form a portion of an annular body when attached to the upper left arm of the living body in cooperation with the frame 22 and the band 12. The support sections 28a, 28b are spaced apart in the longitudinal direction of the band 12. The longitudinal direction is a direction along the extending direction (circumferential direction) with respect to the main body section 14 of the band 12. In the present embodiment, a direction in which the first band section (longitudinal band) 62 described later extends from the main body section 14 is referred to as a first direction D1, and a direction in which the second band section (short band) 64 described later extends from the main body section 14 is referred to as a second direction D2. The rear surface of the frame 22 between the support sections 28a, 28b is planar. The support sections 28a, 28b are inclined with respect to the rear surface of the frame 22 so as to facilitate contact with the upper left arm of the living body. The support section 28a, the rear surface of the frame 22, and the support section 28b are formed in a substantially arc shape as a whole.

The support sections 28a, 28b are formed of a material having elastically deformable flexibility such as a rubber material or a resin material.

The first main body electrode 30 is fixed to the rear surface of the frame 22 of the main body section 14. The second main body electrode 32 is fixed to the support section 28a on one side. The third main body electrode 34 is fixed to the support section 28b on the other side. The first to third main body electrodes 30, 32, 34 are used as a sensor or a transducer for detecting electrical potentials. The first main body electrode 30, the second main body electrode 32, and the third main body electrode 34 are formed in a plate shape with a metal plate or a conductive elastomer having conductivity, for example, and are separated from each other. Here, an example in which the main body section 14 has three main body electrodes 30, 32, 34 will be described. The three main body electrodes 30, 32, 34 are electrically connected to each other and may be formed as being regarded as one electrode, or may be formed as one plate-like electrode.

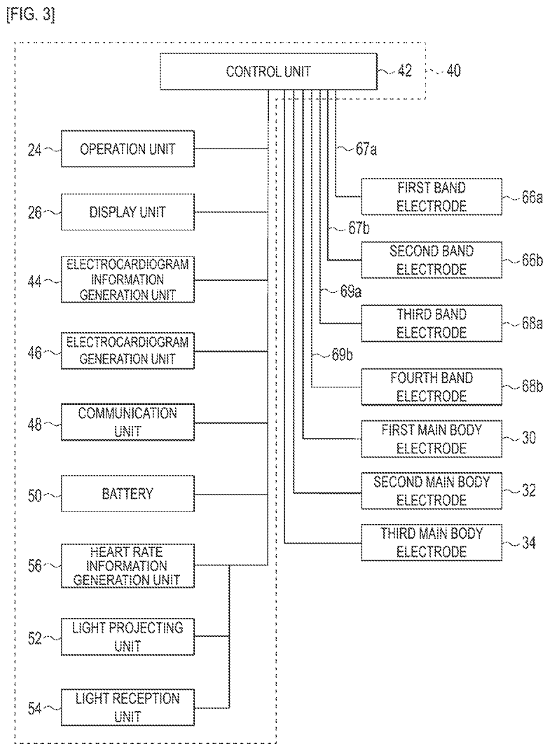

As illustrated in FIG. 3, the main body section 14 includes a control unit 42, an electrocardiogram information generation unit 44, and an electrocardiogram generation unit 46, a communication unit 48, and a battery 50. The main body section 14 further includes a light projecting unit 52, a light reception unit 54, and a heart rate information generation unit 56. The control unit 42, the electrocardiogram information generation unit 44, the electrocardiogram generation unit 46, the communication unit 48, the battery 50, the light projecting unit 52, the light reception unit 54, and the heart rate information generation unit 56 are provided on a control substrate 40 built into the frame 22. The control substrate 40 is further provided with the operation unit 24 and the display unit 26. The operation unit 24, the display unit 26, the first main body electrode 30, the second main body electrode 32, the third main body electrode 34, the electrocardiogram information generation unit 44, the electrocardiogram generation unit 46, the communication unit 48, the light projecting unit 52, the light reception unit 54, and the heart rate information generation unit 56 are, for example, electrically connected to the control unit 42. The control unit 42 is electrically connected to first to fourth band electrodes (biological signal detecting units) 66a, 66b, 68a, 68b, which will be described later. The control unit 42 can control the operation unit 24, the display unit 26, the first main body electrode 30, the second main body electrode 32, the third main body electrode 34, the electrocardiogram information generation unit 44, the electrocardiogram generation unit 46, the communication unit 48, the light projecting unit 52, the light reception unit 54, the heart rate information generation unit 56, and the first to fourth band electrodes 66a, 66b, 68a, 68b, respectively.

The control unit 42 expands a control program stored in a memory such as a ROM by, for example, one or more processors into a RAM according to the power from the battery 50, and executes appropriate processing for the operation unit 24, the display unit 26, the first main body electrode 30, the second main body electrode 32, the third main body electrode 34, the electrocardiogram information generation unit 44, the electrocardiogram generation unit 46, the communication unit 48, the light projecting unit 52, the light reception unit 54, the heart rate information generation unit 56, and the first to fourth band electrodes 66a, 66b, 68a, 68b. Alternatively, in the control unit 42, for example, one or more processors read the program via the network, and execute appropriate processing for the operation unit 24, the display unit 26, the first main body electrode 30, the second main body electrode 32, the third main body electrode 34, the electrocardiogram information generation unit 44, the electrocardiogram generation unit 46, the communication unit 48, the light projecting unit 52, the light reception unit 54, the heart rate information generation unit 56, and the first to fourth band electrodes 66a, 66b, 68a, 68b. By a circuit (for example, ASIC) that realizes one or more functions, the control unit 42 can realize control of the operation unit 24, the display unit 26, the first main body electrode 30, the second main body electrode 32, the third main body electrode 34, the electrocardiogram information generation unit 44, the electrocardiogram generation unit 46, the communication unit 48, the light projecting unit 52, the light reception unit 54, the heart rate information generation unit 56, and the first to fourth band electrodes 66a, 66b, 68a, 68b.

The operation unit 24 has an operation button for inputting instructions such as power ON/OFF, measurement start operation, and measurement stop operation of the main body section 14.

The display unit 26 causes the user to recognize the operation state of the main body section 14 (operation state of the control unit 42) by, for example, emitting light from a light emitting diode (LED). The user can grasp the operation state of the main body section 14 by recognizing the emission color, emission pattern, or the like of the display unit 26 controlled by the control unit 42. The examples of the operation state include a state in which power is turned on by an operation of the operation unit 24, a state in which measurement using the main body section 14 is performed, a state in which an appropriate signal is measured and information is appropriately transmitted to an external apparatus via communication using the communication unit 48. The display unit 26 may be formed as a screen that displays the electrocardiogram information such as the waveform generated by the electrocardiogram generation unit 46 and the heart rate information generated by the heart rate information generation unit 56. An example of the screen is a liquid crystal display or an organic electroluminescent display.

It is also preferable that the operation unit 24 and the display unit 26 are formed as an integrated touch panel. In this case, when the operation unit 24 is operated by the user, the operation unit 24 converts a command into an electrical signal. The display unit 26 can display electrocardiogram information, heart rate information, operation state of the control unit 42, or the like.

The first to fourth band electrodes 66a, 66b, 68a, 68b are used as a sensor or a transducer that obtains electrical potential information, for example. The electrocardiogram information generation unit 44 obtains potential difference information by selectively using a plurality of electrodes from among the first to third main body electrodes 30, 32, 34 and the first to the fourth band electrodes 66a, 66b, 68a, 68b, and generates electrocardiogram information by signal processing. The electrocardiogram generation unit 46 generates information of the electrocardiogram on the basis of the electrocardiogram information generated by the electrocardiogram information generation unit 44. The electrocardiogram information generated by the electrocardiogram information generation unit 44 and the information on the electrocardiogram generated by the electrocardiogram generation unit 46 are transmitted to an external apparatus of the wearable apparatus 10 by the communication unit 48 controlled by the control unit 42 and stored in the external apparatus. Examples of the external apparatus include a data server, a PC, a smartphone, or a tablet terminal. It is also preferable that the electrocardiogram information generated by the electrocardiogram information generation unit 44 and the information on the electrocardiogram generated by the electrocardiogram generation unit 46 are stored in a storage unit (not illustrated) in the main body section 14.

The light projecting unit 52 can emit light having an appropriate wavelength toward the living body through a window section 22a in the first main body electrode 30 on the rear surface illustrated in FIG. 1 of the frame 22. The light reception unit 54 can receive the reflected light of the light emitted from the light projecting unit 52 toward the living body through the window section 22a. The heart rate information generation unit 56 appropriately processes signals to generate heart rate information based on the information obtained by projecting light onto the living body by the light projecting unit 52, obtaining the reflected light from the living body by the light reception unit 54, and performing photoelectric conversion. The heart rate information generated by the heart rate information generation unit 56 is transmitted to the external apparatus of the wearable apparatus 10 by the communication unit 48 controlled by the control unit 42 and stored in the external apparatus. It is also preferable that the heart rate information generated by the heart rate information generation unit 56 are stored in a storage unit (not illustrated) in the main body section 14.

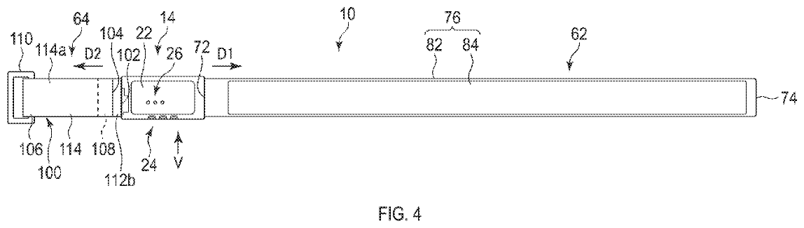

As illustrated in FIGS. 4 to 6, in the present embodiment, the band 12 is extended from the main body section 14 to the two directions D1 and D2 in the opposite direction from each other. The band 12 includes the first band section (first belt section) 62 and the second band section (second belt section) 64. The first band section 62 and the second band section 64 have a band shape (strap shape or strip shape), and extend along the longitudinal direction. Two band electrodes (first and second band electrodes) 66a, 66b are fixed to the first band section 62. The second band electrode 66b fixed to the first band section 62 may be removable from the first band section 62. It is preferable that a plurality of electrodes 66a, 66b are disposed in the first band section 62, but one may be used. Two band electrodes (third and fourth band electrodes) 68a, 68b are fixed to the second band section 64. The fourth band electrode 68b fixed to the second band section 64 may be removable from the second band section 64. It is preferable that a plurality of electrodes 68a, 68b are disposed in the second band section 64, but one may be used.

Note that, for maintenance of the first band section 62, the first band electrode 66a as well as the second band electrode 66b may be removable. Likewise, for maintenance of the second band section 64, the third band electrode 68a as well as the fourth band electrode 68b may be removable.

The main body section 14 is fixed to a desired position on the upper left arm, for example, by using the band 12. The lengths of the first band section 62 and the second band section 64 of the band 12 may be constant regardless of the intended user of the attachment, or may vary depending on the intended user of the attachment. For example, the band 12 having a length assuming an adult, a child, a man, a woman, or the like may be lined up.

Even when it is assumed to be attached to the lower limb instead of the upper limb such as the upper left arm, the lengths of the first band section 62 and the second band section 64 of the band 12 may be the same as those in the case of being attached to the upper limb, or may be changed depending on the envisaged position of attachment.

The first band section 62 has one end 72, the other end 74, and a stretchable section (first band section-side stretchable section) 76. The one end 72 of the first band section 62 is supported by the main body section 14. The first band section 62 is preferably rotatable along the living body, for example at the one end 72. The other end 74 of the first band section 62 is an open end.

The stretchable section 76 of the first band section 62 has first stretchability that allows stretch along the longitudinal direction. The stretchable section 76 having the first stretchability along the longitudinal direction of the first band section 62 is substantially formed as a low stretchable section or a non-stretchable section that hardly stretches along the longitudinal direction. In the present embodiment, the first stretchability of the first band section 62 has substantially the same meaning as low stretchability or non-stretchability. The stretchability in the width direction that is orthogonal to the longitudinal direction of the stretchable section 76 of the first band section 62 is, for example, substantially the same as the first stretchability.

The first band section 62 includes a substrate 82 having a higher stretchability along the longitudinal direction than the first stretchability, and a stretchability adjustment member 84 fixed to the substrate 82.

The stretchability of the substrate 82 along the longitudinal direction per unit length is greater than the first stretchability of the stretchable section 76. The substrate 82 is made of, for example, a fabric. As the substrate 82, for example, a stretch material such as polyurethane, cotton, or a rubber material can be used.

The adjustment member 84 is made of a material having lower stretchability in the longitudinal direction than the substrate 82. The adjustment member 84 is made of, for example, a fabric. As the adjustment member 84, a material having substantially low stretchability or non-stretchability is used.

The substrate 82 and the adjustment member 84 are formed long along the longitudinal direction. The substrate 82 defines the one end 72 and the other end 74 along the longitudinal direction. The length of the adjustment member 84 is slightly shorter than the length of the substrate 82. The adjustment member 84 is fixed between the one end 72 and the other end 74 of the substrate 82. The substrate 82 and the adjustment member 84 are sewn or attached to each other to be fixed. An outer edge of the adjustment member 84 is inside the outer edge of the substrate 82. The substrate 82 is positioned on a living body side when the wearable apparatus 10 is attached to a living body. The adjustment member 84 is positioned on the opposite side to the living body when attached to the living body. The substrate 82 and the adjustment member 84 have flexibility. The substrate 82 and the adjustment member 84 are formed of a material having an electrical insulation property.

Since the adjustment member 84 that is slightly shorter than the entire length of the first band section 62 and has lower stretchability than the substrate 82 is fixed to the substrate 82, the stretchability of the first band section 62 as a whole is lower than the stretchability of the substrate 82. The first stretchability in the longitudinal direction of the first band section 62 is substantially the same as the stretchability of the adjustment member 84 in the longitudinal direction, and has substantially low stretchability or non-stretchability.

For example, a hook and loop fastener is used as the adjustment member 84. The hook and loop fastener is preferably used in which a hook and a loop are mixed in the same surface. The first band section 62 is folded back at an appropriate portion where the substrate 82 and the adjustment member 84 overlap with each other, and is connected by contact between the hook and loop fasteners of the adjustment member 84, so that the length thereof is adjusted. Therefore, the hook and loop fastener of the adjustment member 84 of the first band section 62 is folded back at the ring member 110 toward a position outside the first band section 62 to adjust the length of the first band section 62, and serves as a connecting section that is attachable to and detachable from the outside of the first band section 62.

In the present embodiment, the first band electrode 66a is fixed to the substrate 82 of the first band section 62 at a position close to the second main body electrode 32. The second band electrode 66b is fixed to the substrate 82 of the first band section 62 at a position further away from the second main body electrode 32 than the first band electrode 66a. The first band electrode 66a and the second band electrode 66b are adjacent to each other in the longitudinal direction. The first band electrode 66a and the second band electrode 66b are formed of a conductive metal plate or a conductive elastomer in a plate shape, and hardly extend in the longitudinal direction. The first band electrode 66a is electrically connected to the control unit 42 of the control substrate 40 of the main body section 14 through a wire 67a disposed in the first band section 62. The second band electrode 66b is electrically connected to the control unit 42 of the control substrate 40 of the main body section 14 through the wire 67b disposed in the first band section 62.

As illustrated in FIGS. 4 to 7, the second band section 64 includes a band main body 100 having a band shape and a ring member 110 having a ring shape and provided at a position corresponding to a band end along the longitudinal direction of the second band section 64.

The band main body 100 includes one end 102, the other end 104, a fold-back section 106, and a fixing section (joining section) 108. The band main body 100 is continuous between the one end 102 and the other end 104. The one end 102 of the band main body 100 is supported by the main body section 14. The second band section 64 is preferably rotatable along the living body, for example at the one end 102.

The band end of the second band section 64 is at the most distal position with respect to the one end 102 of the band main body 100 along the second direction D2 in the longitudinal direction. The band main body 100 is folded back at the fold-back section 106 in the ring member 110 at a position corresponding to the band end of the second band section 64. The fold-back section 106 is at the most distal position with respect to the one end 102 of the second band section 64 along the second direction D2 in the longitudinal direction. The other end 104 of the second band section 64 is between the one end 102 and the fold-back section 106 along the longitudinal direction. The fixing section 108 is provided on the other end 104 of the second band section 64. The fixing section 108 is located closer to the main body section 14 than the fold-back section 106 along the longitudinal direction.

A portion between the one end 102 of the band main body 100 and the fold-back section 106 is formed as a first stretchable section (first stretchable region) 112. A portion between the fold-back section 106 and the other end 104 of the band main body 100 is formed as a second stretchable section (second stretchable region) 114. The first stretchable section 112 and the second stretchable section 114 have a band shape (strap shape or strip shape). The fold-back section 106 is provided between the first stretchable section 112 and the second stretchable section 114. The first stretchable section 112 is provided on the inner layer (inner side) of the second band section 64, and the second stretchable section 114 is provided on the outer layer (outer side) of the first stretchable section 112.

The first stretchable section 112 has a first surface 112a and a second surface 112b opposite the first surface 112a. The second stretchable section 114 has a first surface 114a and a second surface 114b opposite the first surface 114a. The first stretchable section 112 is formed continuously at a position close to the one end 102 of the second band section 64. In the first stretchable section 112, the fold-back section 106 and the second stretchable section (second stretchable region) 114 are provided in the path between the most distal end (boundary) 113a along the longitudinal direction from the one end 102 and the fixing section 108. The first stretchable section 112 and the fold-back section 106 are continuous with each other by the boundary 113a. The fold-back section 106 and the second stretchable section 114 are continuous with each other by the boundary 113b. The boundary 113b of the present embodiment is not clearly distinguishable.

In the second band section 64, two stretchable sections 112, 114 are formed in a continuous state by the fold-back section 106, and the two stretchable sections 112, 114 overlap with each other. Specifically, the second stretchable section 114 overlaps with the first stretchable section 112 on the second surface 112b side of the first stretchable section 112. Thus, the second surface 112b of the first stretchable section 112 and the second surface 114b of the second stretchable section 114 face each other or come into contact with each other. The fixing section 108 fixes the second surface 112b of the first stretchable section 112 and the second surface 114b of the second stretchable section 114.

The first stretchable section 112 is located in the second direction D2 along the longitudinal direction with respect to the main body section 14. It is preferable that the first stretchable section 112 has first stretchability along the longitudinal direction. The first stretchability is, as described above, low stretchability or non-stretchability, and substantially does not allow stretching. The third and fourth band electrodes 68a, 68b are provided on the first surface 112a of the first stretchable section 112.

The third and fourth band electrodes 68a, 68b are formed of a conductive metal plate or a conductive elastomer in a plate shape, and hardly stretch in the longitudinal direction and the direction orthogonal to the longitudinal direction. The third band electrode 68a is electrically connected to the control unit 42 of the control substrate 40 of the main body section 14 through a wire 69a disposed in the second band section 64. The fourth band electrode 68b is electrically connected to the control unit 42 of the control substrate 40 of the main body section 14 through a wire 69b disposed in the first stretchable section 112 of the second band section 64. The wires 69a, 69b are provided, for example, between the first surface 112a and the second surface 112b of the first stretchable section 112. The wires 69a, 69b are preferably not exposed to the outside of the first stretchable section 112.

The fold-back section 106 folds back the band main body 100 at the ring member 110. Thus, the band main body 100 is folded back by the fold-back section 106, so that the first stretchable section 112 and the second stretchable section 114 overlap with each other. The fold-back section 106 is located on the second direction D2 side along the longitudinal direction with respect to the first stretchable section 112. The fold-back section 106 has second stretchability that allows greater stretch along the longitudinal direction per unit length than the first stretchability of the first stretchable section 112. The fold-back section 106 is at a position with a constant distance from the electrode 68b. There is the boundary 113a between the fold-back section 106 and the electrode 68b.

The stretchability in the width direction orthogonal to the longitudinal direction of the first stretchable section 112 of the second band section 64 and the stretchability in the width direction orthogonal to the longitudinal direction of the second stretchable section 114 of the second band section 64 are, for example, substantially the same as the first stretchability.

The second band section 64 includes a substrate (base section) 122 that is on the upper left arm side (living body side) when attached to the upper left arm of the living body, and a stretchability adjustment member 124 that is on the outer side away from the upper left arm. The substrate 122 and the adjustment member 124 are formed long along the longitudinal direction. The substrate 122 and the adjustment member 124 have flexibility. The substrate 122 and the adjustment member 124 are formed of a material having an electrical insulation property. The substrate 122 and the adjustment member 124 are sewn or attached to each other to be fixed. An outer edge of the adjustment member 124 is inside the outer edge of the substrate 122. The substrate 122 is made of, for example, a fabric. As the substrate 122, for example, a stretch material such as polyurethane, cotton, or a rubber material can be used. The substrate 122 has third stretchability that allows greater stretch along the longitudinal direction than the first stretchability. The adjustment member 124 is made of a material having lower stretchability in the longitudinal direction than the substrate 122. The adjustment member 124 is made of, for example, a fabric. The adjustment member 124 has fourth stretchability that allows lower stretch along the longitudinal direction than the third stretchability. The fourth stretchability is, for example, similar to the first stretchability, low stretchability or non-stretchability.

The adjustment member 124 is located between the one end 102 and the position corresponding to the fold-back section 106 along the longitudinal direction of the substrate 122. The adjustment member 124 is not disposed at a position corresponding to the fold-back section 106.

The stretchability in the longitudinal direction of the region where the adjustment member 124 having the fourth stretchability is fixed to the substrate 122 having the third stretchability is substantially the same as the fourth stretchability in the longitudinal direction of the adjustment member 124. The fourth stretchability is substantially the same as the first stretchability. Therefore, a region where the adjustment member 124 having low stretchability along the longitudinal direction is fixed to the substrate 122 is formed as the first stretchable section 112 having the first stretchability. In this manner, the substrate 122 and the adjustment member 124 are sewn or attached to each other, so that the stretchability in the longitudinal direction of the first stretchable section 112 of the second band section 64 is adjusted to the first stretchability.

The stretchability in the longitudinal direction of the region where the adjustment member 124 is not fixed to the substrate 122 is substantially the same as the third stretchability in the longitudinal direction of the substrate 122. The third stretchability is substantially the same as the second stretchability. Therefore, a region where the adjustment member 124 is not fixed to the substrate 122 along the longitudinal direction is formed as a second stretchable section 114 having second stretchability.

The fold-back section 106 of the second band section 64 is formed similarly to the second stretchable section 114 (substrate 122) having higher stretchability along the longitudinal direction than the first stretchability. The fold-back section 106 does not include the adjustment member 124. Therefore, similarly to the second stretchable section 114, the fold-back section 106 is more stretchable in the longitudinal direction than the first stretchable section 112.

The fixing section 108 joins the second surface 114b of the second stretchable section 114 including, for example, the other end 104 of the second band section 64, and the second surface 112b on the outer side of the first stretchable section 112 of the second band section 64. Therefore, the fixing section 108 joins the first stretchable section 112 and the second stretchable section 114 to each other at a position closer to the main body section 14 than the fold-back section 106 on the second direction D2 side along the longitudinal direction with respect to the main body section 14.

For example, an annular ring member 110 is supported by the fold-back section 106. The ring member 110 is used in connecting the first band section 62 and the second band section 64. The ring member 110 is formed in, for example, a rectangular annular shape. The ring member 110 is preferably formed of a material having an electrical insulation property, for example. The ring member 110 is movable along the longitudinal direction by the second stretchability of the second stretchable section 114.

The operation of the wearable apparatus 10 will be described with reference to the case where the wearable apparatus 10 is attached to the upper left arm UA of the living body.

As illustrated in FIGS. 1, 2, and 8, the user passes the other end 74 of the first band section 62 through the ring member 110, which is supported by the fold-back section 106 of the second band section 64, from the inner side to the outer side, and forms a loop with the band 12 and the main body section 14. The user brings the first to third main body electrodes 30, 32, 34 of the main body section 14, the first to fourth band electrodes 66a, 66b, 68a, 68b into direct contact with the skin of the upper left arm UA of the user. In this state, the user determines the positions of the electrodes 30, 32, 34 of the main body section 14, the electrodes 66a, 66b of the first band section 62, and the electrodes 68a, 68b of the second band section 64 with respect to the upper left arm UA.

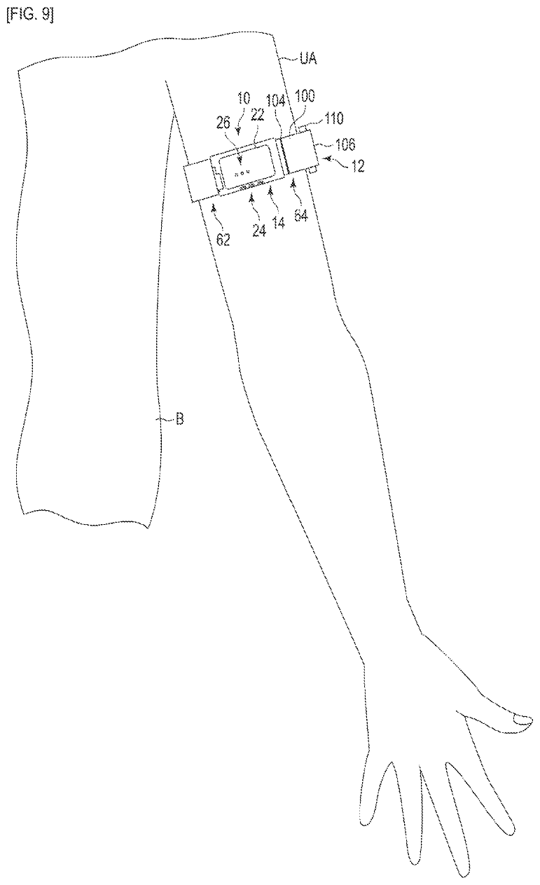

As illustrated in FIG. 9, it is preferable that the attachment of the wearable apparatus 10 with respect to the upper left arm UA of the user is such that the main body section 14 faces the front (anterior) of the user when the body B of the user is located in a normal anatomical position, for example.

The minimum and maximum values of the applicable circumferential length of the wearable apparatus 10 to the upper left arm UA are predetermined. The user adjusts the length of the stretchable section 76 of the first band section 62 depending on the outer circumferential length of the upper left arm UA, and adjusts the position of the fold-back section 76a with respect to the ring member 110. In other words, in attaching the band 12 to the upper left arm UA, the user performs initial adjustment of the circumferential length of the band 12 by adjusting the position of the inside of the fold-back section 76a and the end section 74 of the first band section 62. The first band section 62 is folded back at a desired position of the first band section 62 and the ring member 110, and the stretchability adjustment members (connecting sections) 84 of the hook and loop fastener are fastened to each other. Thus, the position of the first band section 62 with respect to the upper left arm UA of the user is fixed. The user fixes the position of the fold-back section 76a and the end section 74 of the first band section 62 in a state in which the user feels that the fixed state of the band 12 is neither too loose nor too tight by himself/herself when the user is located in a normal anatomical position. In the present embodiment, since the initial length of the second band section 64 is predetermined by the fixing section 108, the wearable apparatus 10 is maintained in a state of being attached to the upper left arm UA of the user.

When the user fixes the first band section 62 via the ring member 110 and the wearable apparatus 10 becomes an annular body, the second and third main body electrodes 32, 34 are pressed against the skin of the upper left arm UA, and the support sections 28a, 28b elastically deform. Thus, the electrodes 32, 34 are adhered to the skin of upper left arm UA. The first main body electrode 30 is located between the second and third main body electrodes 32, 34, and the second and third main body electrodes 32, 34 are pressed against and adhered to the skin of the upper left arm UA, and are adhered to the skin of the upper left arm UA.

Similarly, the first and second band electrodes 66a, 66b provided in the first band section 62 having flexibility are adhered to the skin of the upper left arm UA, and the third and fourth band electrodes 68a, 68b provided in the second band section 64 having flexibility are adhered to the skin of the upper left arm UA.

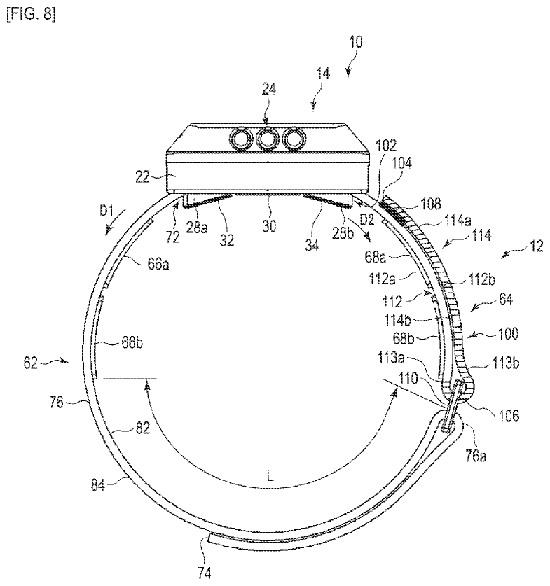

The reference sign L in FIG. 8 is the circumferential length between the electrode 66b of the first band section 62 and the ring member 110. The length L is varied depending on the thickness or attachment site of the upper left arm UA of the user. For example, when the outer diameter of the upper left arm UA of the user is relatively small and the length L is short, the electrodes 66b, 68b come close to each other in a state of being separated from each other, and the electrodes 30, 32, 34, 66a, 66b, 68a, 68b come into contact with the skin so as to substantially cover the upper left arm UA of the user with the electrodes 30, 32, 34, 66a, 66b, 68a, 68b. At this time, the path from the electrode 66b to the electrode 68b through the ring member 110 is shorter than the path from the electrode 66b to the electrode 68b through the main body section 14. As an example, the total contact area of the electrodes 30, 32, 34, 66a, 66b, 68a, 68b of the contact surface of the user's upper left arm UA with respect to the wearable apparatus 10 may be larger than the contact area where the first band section 62 and the second band section 64 themselves contact the user's upper left arm UA. The outer diameter of the attachment position of the upper left arm UA when L=0 is the minimum value of the applicable circumferential length of the wearable apparatus 10.

When the outer diameter of the upper left arm UA of the user is large and the length L is large, the electrodes 66b, 68b are separated from each other. At this time, the path from the electrode 66b to the electrode 68b through the main body section 14 may be shorter than the path from the electrode 66b to the electrode 68b through the ring member 110.

The user appropriately operates the operation unit 24 in a state where the wearable apparatus 10 is appropriately attached to the upper left arm UA of the user. When the main body section 14 is powered on, the control unit 42 starts communication with the external apparatus by the communication unit 48.

Under the control of the control unit 42, the main body section 14 detects signals (electrocardiographic signals) corresponding to the potentials of the skin surfaces of the upper arm UA using the first to third main body electrodes 30, 32, 34, and the first to fourth band electrodes 66a, 66b, 68a, 68b. The first to third main body electrodes 30, 32, 34, and the first to fourth band electrodes 66a, 66b, 68a, 68b output signals corresponding to electrical potentials to the electrocardiogram information generation unit 44. The electrocardiogram information generation unit 44 selects one electrode selected from a plurality of electrodes including the first to third main body electrodes 30, 32, 34 and the first to fourth band electrodes 66a, 66b, 68a, 68b, set the selected electrode as a reference electrode, calculates the potential difference between the detection potential of the reference electrode and the detection potentials of the remaining electrodes of the plurality of electrodes, and generates information about the electrocardiographic signal based on the maximum negative value and the maximum positive value among the calculation results. The electrocardiogram information generation unit 44 obtains information about the electrocardiographic signal based on the detected potential of the selected electrode. The electrocardiogram information generation unit 44 can obtain information from a plurality of electrode pairs, not only one electrode pair (paired electrodes). In general, the larger the distance between the paired electrodes, the larger the potential difference. Since the distance between the paired electrodes varies depending on the thickness (circumferential length) of the arm, a combination of the paired electrodes having a large potential difference is selected each time. The electrocardiogram information generation unit 44 generates an electrocardiographic signal using a stronger signal among the signals obtained from the plurality of electrode pairs. The electrocardiogram generation unit 46 generates an electrocardiogram waveform based on the information obtained by the electrocardiogram information generation unit 44.

The heart rate information generation unit 56 obtains heart rate information based on the light projection from the light projecting unit 52 to the upper left arm UA and the detection state of the reflected light from the upper left arm UA.

In this manner, the main body section 14 of the wearable apparatus 10 measures the electrocardiographic signal and the heart rate of the user to obtain the respective pieces of information. The user can easily recognize the state of his/her heart by looking at a display screen of a PC, a smartphone, a tablet terminal, or the like connected by the communication unit 48.

In some cases, it is preferable that the measurement of the electrocardiographic signal and the heart rate measurement is performed over several hours until an appropriate time has elapsed. Examples of the measurement of the electrocardiographic signal and the heart rate measurement over several hours include the measurement during active hours or the measurement during sleep.

When the user to whom the wearable apparatus 10 is attached moves his/her left arm, for example, bends and stretches his/her left arm, the size (circumferential length) of the outer circumference of the upper left arm UA may vary according to the movement of the left arm. In general, when a user stretches his/her arm, the circumferential length becomes relatively small, and when the user bends his/her arm, the circumferential length becomes larger than that in a state where the arm is stretched.

The electrode-equipped band 12 includes, in one second band section 64, a first stretchable section 112, a second stretchable section 114 having stretchability higher than that of the first stretchable section 112, and the fold-back section 106 between the first stretchable section 112 and the second stretchable section 114. In the second band section 64, a continuous region from the boundary 113a between the first stretchable section 112 and the fold-back section 106 to the fixing section 108 has second stretchability. The second stretchable section 114 stretches in the longitudinal direction (circumferential direction) in accordance with the movement of the upper left arm (living body) UA at the position where the band 12 is attached, and the fold-back section 106 that is continuous with the second stretchable section 114 and has the second stretchability stretches in the longitudinal direction. The relative position between the fold-back section 106 and the ring member 110 is varied in accordance with the stretch of the second stretchable section 114 and the fold-back section 106 in the longitudinal direction. Thus, the circumferential length of the band 12 is adjusted by the second band section 64.

The boundary 113a between the first stretchable section 112 of the second band section 64 having the low stretchability or non-stretchability, and the fold-back section 106 having appropriate stretchability, is on the side coming into close contact with the upper left arm UA of the user. Then, a region from the boundary 113a on the upper left arm UA side of the user to the fixing section 108 in the vicinity of the other end 104 of the second band section 64 through the fold-back section 106 can be continuously formed as a region having the second stretchability. As described above, the fixing section 108 is located closer to the main body section 14 than the fold-back section 106 along the longitudinal direction. Thus, the length along the longitudinal direction of the second stretchable section 114 can be made relatively long. Therefore, in the second band section 64, the stretchable region can be made relatively large along the longitudinal direction.

Thus, in accordance with the movement of the left arm, the fold-back section 106 and the second stretchable section 114 stretch along the longitudinal direction of the band 12. In this manner, due to the stretchability along the longitudinal direction (circumferential direction) of the fold-back section 106 of the second band section 64, the band 12 will easily follow the movement of the upper left arm UA. Therefore, it is possible to prevent the user from feeling uncomfortable in a state where the wearable apparatus 10 is attached to the upper left arm UA of the user.

In the first band section 62, the electrodes 66a, 66b are provided at a portion having the first stretchability. That is, in the first band section 62, the electrodes 66a, 66b are provided in the first band section 62 having low stretchability or non-stretchability that allows lower stretch than the second stretchable section 114 of the second band section 64. In the second band section 64, the electrodes 68a, 68b are provided in the first stretchable section 112 that is adjacent to the main body section 14 and has the first stretchability. That is, in the second band section 64, the electrodes 68a, 68b are provided in the first stretchable section 112 having low stretchability or non-stretchability that allows lower stretch than the second stretchable section 114 of the second band section 64. Thus, the wearable apparatus 10 maintains the positional relationship between the upper left arm UA and the electrodes 66a, 66b, 68a, 68b in a fixed state with respect to the user to whom the band 12 is attached. After the user has moved his/her left arm appropriately, the main body section 14 faces the anterior of the user when the user is located in a normal anatomical position. When the user moves his/her left arm, the electrodes 30, 32, 34, 66a, 66b, 68a, 68b are maintained in contact with the upper left arm UA of the user. Thus, the wearable apparatus 10 is capable of detecting stable electrocardiographic signals using the electrodes 66a, 66b, 68a, 68b, and detection of the heart rate signal using the light projecting unit 52 and the light reception unit 54.

Note that, a site of the first band section 62 in which the wires 66a, 66b that connect the electrodes 67a, 67b and the control unit 42 are accommodated is formed of a material having low stretchability or non-stretchability. Similarly, among the second band sections 64, a site where the wires 69a, 69b that connect the electrodes 68a, 68b and the control unit 42 are accommodated is formed of a material having low stretchability or non-stretchability. Therefore, even when the user appropriately moves the left arm, a force that expands the wires 67a, 67b, 69a, 69b is unlikely to be applied to the wires 67a, 67b, 69a, 69b.

After obtaining the electrocardiogram information and the information and the heart rate information for a preset time, the control unit 42 automatically ends the detection of the information related to the electrocardiographic signal and the heart rate information. The control unit 42 automatically turns the power source of the main body section 14 to OFF after the detection of the information related to the electrocardiographic signal and the heart rate information. Thereafter, the user releases the fixing of the first band section 62 of the wearable apparatus 10, and removes the wearable apparatus 10 from the upper left arm UA.

The wearable apparatus 10 is maintained without being removed while the circumferential length changes in accordance with the operation of the left arm from the start to end of the information related to the electrocardiographic signal and the detection of the heart rate information. At this time, each of the electrodes 30, 32, 34, 66a, 66b, 68a, 68b is maintained in contact with the skin of the upper left arm UA. After the upper left arm UA is appropriately moved, when the user is located in a normal anatomical position, the main body section 14 faces the anterior of the user. Thus, after the wearable apparatus 10 is attached to the user, the wearable apparatus 10 can stably detect information related to the electrocardiographic signal and the heart rate information from the start to end of the user's electrocardiogram and heart rate test.

The wearable apparatus 10 is used in this manner.

The wearable apparatus 10 can be used not only for measurement for a relatively long time of several hours, such as during sleep or during activity, but also for measurement for a relatively short time, such as within one minute or several minutes. Thus, the band 12 can be used even in a case where a state in which the main body section 14 is attached to the living body is maintained in a relatively short time, for example, within several minutes.

The example in which the wearable apparatus 10 is mounted on the upper left arm UA of the user has been described. The wearable apparatus 10 may be mounted on, for example, a wrist or a lower limb of the user. The lower limb includes a thigh, a lower leg, or an ankle.

In the wearable apparatus 10, the band 12 can be used for the wearable apparatus 10 that is attached to a limb to obtain biological information, such as myoelectric information, in addition to information about an electrocardiographic signal. In a case where a biological signal is detected, for example, a pulse wave sensor may be used instead of the electrode 30 or the light projecting unit 52 and the light reception unit 54. In this case, the wearable apparatus 10 calculates a pulse transit time (PTT) from a time difference between an R-wave peak time of the information on the electrocardiographic signal and a rising point of the pulse wave information obtained by using the pulse wave sensor, and estimates the blood pressure.

Although an example in which the electrodes (transducers) 30, 32, 34, 66a, 66b, 68a, 68b of the wearable apparatus 10 are used for potential detection has been described, it is also preferable to use them as pads for low-frequency treatment apparatus or pads for feeble current treatment apparatus, for example. Therefore, it is also preferable that the wearable apparatus 10 according to the present embodiment is attached to the living body not only to detect the biological signal, but also to treat the living body. That is, the band 12 can be used to maintain a state in which the main body section 14 having the function of treating the living body is attached to the living body.

The user may attach the wearable apparatus 10 to the chest, abdomen, or waist by appropriately adjusting the length of the first band section 62 and the second band section 64 of the band 12 described above and the second stretchability of the second stretchable section 114.

The stretchable section 76 of the first band section 62 may have higher stretchability than when it has low stretchability or non-stretchability. At this time, the stretchability of the stretchable section 76 of the first band section 62 is lower than the second stretchability along the longitudinal direction of the second stretchable section 114 of the second band section 64. Similarly, the first stretchability of the first stretchable section 112 of the second band section 64 is lower than the second stretchability of the second stretchable section 114. Therefore, the first stretchability may not be limited to low stretchability or non-stretchability.

As described above, according to the present embodiment, it is possible to provide the electrode-equipped band 12 and the wearable apparatus 10 capable of suppressing the occurrence of discomfort to a user in a state of being attached to an appropriate position.

In the present embodiment, the structure of the second stretchable section 114 of the second band section 64 can be applied to a portion between the electrode 66b of the first band section 62 and the ring member 110.

### Second Embodiment

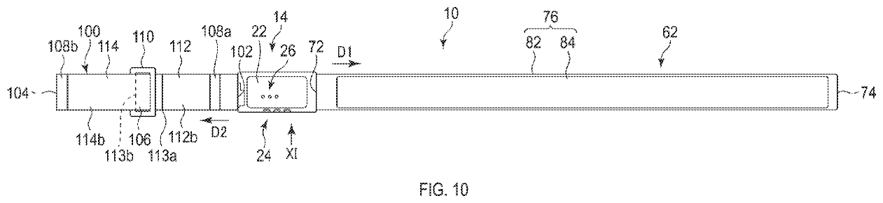

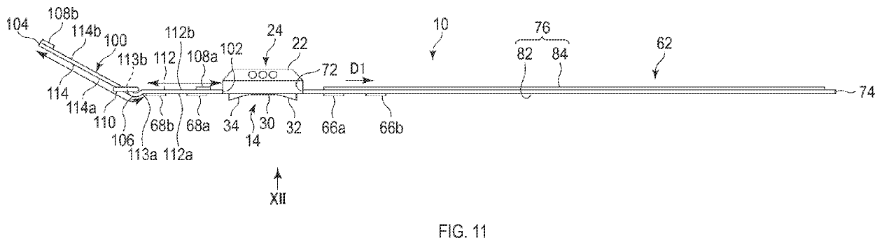

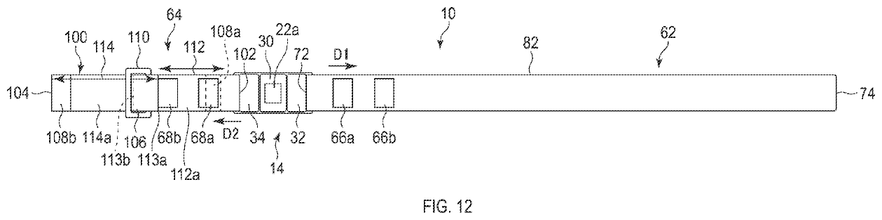

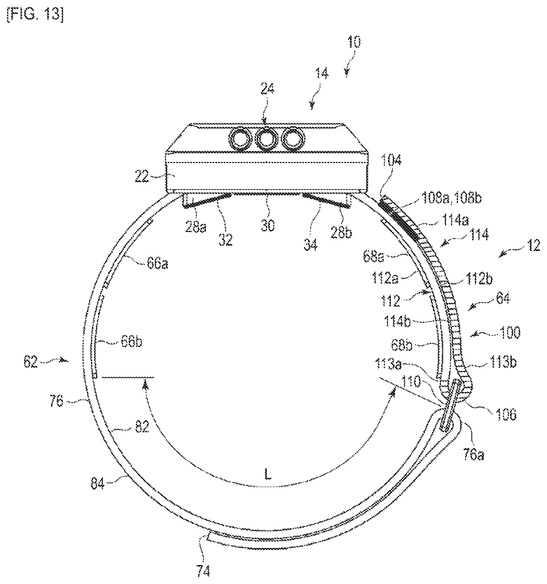

A second embodiment will be described with reference to FIGS. 10 to 13. The second embodiment is a modified example of the first embodiment, and the same reference signs are given to members having the same components or the same functions as those described in the first embodiment, and detailed description thereof is omitted.

As illustrated in FIGS. 10 to 13, the fixing section (joining section) 108 includes a first fixing member (joining section) 108a and a second fixing member (joining section) 108b. In the present embodiment, the first fixing member 108a is fixed to the second surface 112b on the outer side of the first stretchable section 112 between the one end 102 of the band main body 100 of the second band section 64 and the fold-back section 106. The second fixing member 108b is fixed to the second surface 114b of the second stretchable section 114 of the other end 104 of the second band section 64. The first fixing member 108a is, for example, a hook or loop surface of the hook and loop fastener. The second fixing member 108b is the loop surface of the hook and loop fastener when the first fixing member 108a is the hook surface of the hook and loop fastener. The second fixing member 108b is the hook face of the hook and loop fastener when the first fixing member 108a is the loop surface of the hook and loop fastener. The hook and loop fastener of the first fixing member 108a and the second fixing member 108b is preferably used in which the hook and the loop are mixed in the same surface. Therefore, the user can attach and detach the first fixing member 108a and the second fixing member 108b.

The fixed position (fixed state) of the first fixing member 108a and the second fixing member 108b can be adjusted within the range of the length in the longitudinal direction of the first fixing member 108a and the second fixing member 108b. The position of the fold-back section 106 of the band main body 100 is adjusted by adjusting the relative position of the first fixing member 108a and the second fixing member 108b. Therefore, the length of the second band section 64 is also adjustable, although by a small amount as compared with the length adjustment of the first band section 62.

When the user attaches the wearable apparatus 10 to the upper left arm UA of the user, for example, the first band section 62 is fixed as described in the first embodiment in a state where the first fixing member 108a and the second fixing member 108b of the second band section 64 are fixed at the temporary position. Thereafter, if necessary, the user re-fixes the first fixing member 108a and the second fixing member 108b of the second band section 64 in a more comfortable state when moving the left arm.

The position of the fold-back section 106 and the ring member 110 at the most distal position in the second direction D2 of the second band section 64 with respect to the one end 102 of the second band section 64 varies depending on the relationship between the first fixing member 108a and the second fixing member 108b. Therefore, when the wearable apparatus 10 is attached to the user, the circumferential length of the wearable apparatus 10 can be adjusted by the second band section 64, although the adjustment range is smaller than the length adjustment range of the first band section 62. Since the fold-back section 106 has higher stretchability than the low stretchability or non-stretchability of the first stretchable section 112, when the left arm is moved, for example, the left arm is bent and stretched, the fold-back section 106 stretches following the movement. At this time, since the length of the second band section 64 in the longitudinal direction can be adjusted, it is possible to prevent the user from feeling uncomfortable as compared with the case described in the first embodiment while maintaining a suitable attachment state of the wearable apparatus 10 to the user.

According to the present embodiment, it is possible to provide the electrode-equipped band 12 and the wearable apparatus 10 capable of suppressing the occurrence of discomfort to a user in a state of being attached to an appropriate position.

### Third Embodiment

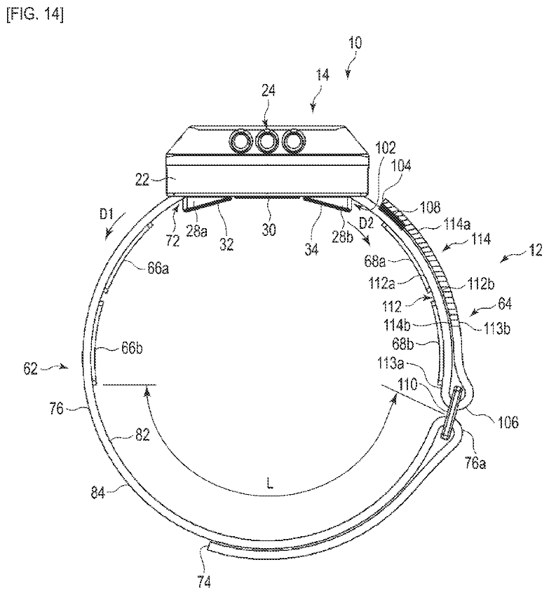

A third embodiment will be described with reference to FIG. 14. The third embodiment is a modified example of the first embodiment and the second embodiment. The same reference signs are given to members having the same components or the same functions as those described in the first embodiment and the second embodiment, and detailed description thereof is omitted.

As illustrated in FIG. 14, the fold-back section 106 of the present embodiment has the same configuration as that of the first stretchable section 112. Thus, the fold-back section 106 according to the present embodiment has the first stretchability.

For example, when the user stretches the left arm in a state where the wearable apparatus 10 is attached to the upper left arm UA of the user, the circumferential length of the left arm becomes relatively small, and when the user bends the left arm, the circumferential length of the left arm becomes larger than that in the state where the arm is stretched.

When the user bends the left arm and the circumferential length of the left arm increases, the second stretchable section 114 extends in the longitudinal direction in the path between the fold-back section 106 and the fixing section 108. By extension of the second stretchable section 114, the path length of the band main body 100 between the one end 102 and the other end 104 of the second band section 64 extends. Therefore, the relative positional relationship of the ring member 110 with respect to the fold-back section 106 is shifted, and the distance between the one end 102 of the second band section 64 and the fold-back section 106 is increased.

In this manner, the fold-back section 106 may have the first stretchability.

### Fourth Embodiment

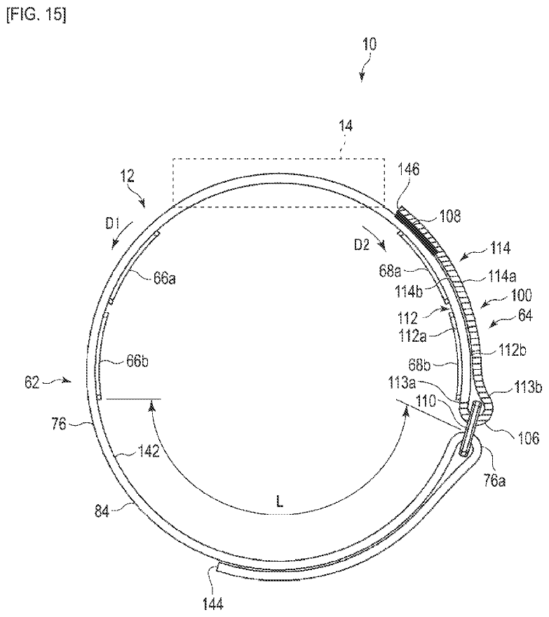

A fourth embodiment will be described with reference to FIG. 15. The fourth embodiment is a modified example of the first embodiment to the third embodiment. The same reference signs are given to members having the same components or the same functions as those described in the first embodiment to the third embodiment, and detailed description thereof is omitted.

In the present embodiment, the first band section 62 and the second band section 64 of the band 12 of the wearable apparatus 10 are continuous. The main body section 14 is removable with regard to the band 12.

The one end 72 of the first band section 62 described in the first embodiment to the third embodiment does not exist. Similarly, the one end 102 of the second band section 64 does not exist. The substrate 82 of the first band section 62 and the substrate 122 of the second band section 64 are formed, for example, as one substrate (base section) 142 in the present embodiment. The substrate 142 has a first end section 144 corresponding to the other end 74 of the first band section 62 and a second end section 146 corresponding to the other end 104 of the second band section 64.

The structure of the fixing section 108 of the second band section 64 of the fourth embodiment may include the first fixing member 108a and the second fixing member 108b described in the second embodiment.

For example, the main body section can be attached and detached at a position indicated by a broken line indicated by reference sign 14 of the band 12. The main body section 14 may have an appropriate test function as described in the first embodiment, an appropriate treatment function, or a combination of the test function and the treatment function. In addition to a medical apparatus, a general apparatus that is not a medical apparatus can be used as the main body section 14.

According to the present embodiment, as described in the first embodiment to the third embodiment, it is possible to provide the electrode-equipped band 12 capable of suppressing the occurrence of discomfort to a user in a state of being attached to an appropriate position.

For example, it is preferable that the electrode-equipped band 12 has a structure in which the wires 67a, 67b, 69a, 69b, and the electrodes 66a, 66b, 68a, 68b can be removed. In this case, in a state where the wires 67a, 67b, 69a, 69b, and the electrodes 66a, 66b, 68a, 68b are removed from the band 12, cleaning, disinfecting, or the like of the band 12 can be performed.

In the fourth embodiment, an example is described in which the band 12 and the main body section 14 are removable. The wearable apparatus 10 may be configured such that the first band section 62 and the second band section 64 are integrated, and the main body section 14 cannot be easily removed from the band 12.

According to the present embodiment, as described in the first embodiment to the third embodiment, it is possible to provide the wearable apparatus 10 and the electrode-equipped band 12 capable of suppressing the occurrence of discomfort to a user in a state of being attached to an appropriate position.

As described above, according to the first embodiment to the fourth embodiment described above, it is possible to provide the electrode-equipped band 12 and the wearable apparatus 10 capable of suppressing the occurrence of discomfort to a user in a state of being attached to an appropriate position.

Note that the present invention is not limited to the embodiments described above, various embodiments and modifications within the spirit of invention are possible. The embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### REFERENCE SIGNS LIST

10 Wearable apparatus
12 Electrode-equipped band
14 Main body section
22 Frame
30 First main body electrode
32 Second main body electrode
34 Third main body electrode
62 First band section
64 Second band section
66a First band electrode
66b Second band electrode
68a Third band electrode
68b Fourth band electrode
76 Stretchable section
76a Fold-back section
81 Substrate
84 Stretchability adjustment member
100 Band main body section
106 Fold-back section
108 Fixing section (joining section)
110 Ring member
112 First stretchable section
114 Second stretchable section
122 Substrate
124 Stretchability adjustment member

What is claimed is:

1. An electrode-equipped band comprising:
a first band section having a band shape and extending along a longitudinal direction; and
a second band section provided in a first direction along the longitudinal direction with respect to the first band section and extending along the longitudinal direction, wherein
the second band section includes:
a first stretchable section having a band shape, having a first surface and a second surface opposite to the first surface, having first stretchability along the longitudinal direction, and having an electrical insulation property;
an electrode being conductive and provided on the first surface of the first stretchable section;
a second stretchable section having a band shape, overlapping with the first stretchable section on a side of the second surface of the first stretchable section, the second stretchable section having second stretchability that allows greater stretch along the longitudinal direction per unit length than the first stretchability of the first stretchable section;
a ring member having a ring shape, provided at a position corresponding to a band end along the longitudinal direction of the second band section, and connectable to the first band section;
a fold-back section provided between the first stretchable section and the second stretchable section, the fold-back section being folded back at the ring member so that the first stretchable section and the second stretchable section overlap with each other; and
a joining section configured to join the second surface of the first stretchable section and the second stretchable section.

2. The electrode-equipped band according to claim 1, wherein,
the first stretchable section includes:
a base section having third stretchability that allows greater stretch along the longitudinal direction than the first stretchability, the base section forming the first surface; and
a stretchability adjustment member fixed to the base section and having fourth stretchability that allows lower stretch along the longitudinal direction than the third stretchability, the stretchability adjustment member being configured to adjust stretchability of the first stretchable section along the longitudinal direction so that the first stretchable section has the first stretchability, the stretchability adjustment member forming the second surface.

3. The electrode-equipped band according to claim 1, wherein
the first stretchable section has low stretchability or non-stretchability along the longitudinal direction.

4. The electrode-equipped band according to claim 1, wherein
the fold-back section has the first stretchability in the longitudinal direction.

5. The electrode-equipped band according to claim 1, wherein
the fold-back section has the second stretchability in the longitudinal direction.

6. The electrode-equipped band according to claim 1, wherein
the first band section is folded back at the ring member toward a position outside the first band section to adjust a length of the first band section, and has a detachable connecting section on the outside of the first band section.

7. The electrode-equipped band according to claim 1, wherein
the first stretchable section of the second band section includes a wire electrically connecting the electrode and a main body section provided in the electrode-equipped band.

8. A wearable apparatus comprising:
the electrode-equipped band according to claim 1; and
a main body section provided between the first band section and the second band section of the electrode-equipped band and incorporating a control substrate electrically connected to the electrode.

9. The electrode-equipped band according to claim 2, wherein
the first stretchable section has low stretchability or non-stretchability along the longitudinal direction.

10. The electrode-equipped band according to claim 2, wherein
the fold-back section has the first stretchability in the longitudinal direction.

11. The electrode-equipped band according to claim 3, wherein
the fold-back section has the first stretchability in the longitudinal direction.

12. The electrode-equipped band according to claim 9, wherein
the fold-back section has the first stretchability in the longitudinal direction.

13. The electrode-equipped band according to claim 2, wherein
the fold-back section has the second stretchability in the longitudinal direction.

14. The electrode-equipped band according to claim 3, wherein
the fold-back section has the second stretchability in the longitudinal direction.

15. The electrode-equipped band according to claim 9, wherein
the fold-back section has the second stretchability in the longitudinal direction.

16. The electrode-equipped band according to claim 2, wherein
the first band section is folded back at the ring member toward a position outside the first band section to adjust a length of the first band section, and has a detachable connecting section on the outside of the first band section.

17. The electrode-equipped band according to claim 3, wherein
the first band section is folded back at the ring member toward a position outside the first band section to adjust a length of the first band section, and has a detachable connecting section on the outside of the first band section.

18. The electrode-equipped band according to claim 4, wherein
the first band section is folded back at the ring member toward a position outside the first band section to adjust a length of the first band section, and has a detachable connecting section on the outside of the first band section.

19. The electrode-equipped band according to claim 9, wherein
the first band section is folded back at the ring member toward a position outside the first band section to adjust a length of the first band section, and has a detachable connecting section on the outside of the first band section.

20. The electrode-equipped band according to claim 10, wherein
the first band section is folded back at the ring member toward a position outside the first band section to adjust a length of the first band section, and has a detachable connecting section on the outside of the first band section.
